(19) United States
(12) Patent Application Publication
(10) Pub. No.: US 2006/0177517 A1
(43) Pub. Date: Aug. 10, 2006

(54) Antiinflammtory agent and antiinflammatory medical material

(76) Inventors: Toshikazu Shiba, Tokyo (JP); Toshie Shiba, Nagano (JP); Mimoru Yamaoka, Nagano (JP); Takashi Uematsu, Nagano (JP); Yoshiharu Takahashi, Nagano (JP); Hitoshi Tanaka, Nagano (JP); Takao Kogo, Hokkaido (JP); Masanobu Shindo, Hokkaido (JP)

Correspondence Address: SUGHRUE MION, PLLC, 2100 PENNSYLVANIA AVENUE, N.W., SUITE 800, WASHINGTON, DC, 20037, US

(21) Appl. No.: 10/546,922

(22) PCT Filed: Feb. 25, 2004
(86) PCT No.: PCT/JP04/02162

§ 371 Date: Aug. 25, 2005

(30) Foreign Application Priority Data
Feb. 26, 2003 (JP) 2003-048460

(51) Int. Cl. A61K 33/42

(52) U.S. Cl. 424/601

(57) ABSTRACT

An object of the present invention is to provide an anti-inflammatory agent and an anti-inflammatory medical material, which have a good anti-inflammatory effect and high safety. The present invention relates to an anti-inflammatory agent and an anti-inflammatory medical material, which contain as an active ingredient a polyphosphoric acid that is particularly 1 type of liner phosphoric acid represented by a general formula Hₙ₊₂(PₙO₃ₙ₊₁) (wherein n denotes an integer between 3 and 800) or a mixture of 2 or more types of such linear phosphoric acid.

### TECHNICAL FIELD

The present invention relates to an anti-inflammatory agent and an anti-inflammatory medical material, which contain a polyphosphoric acid as an active ingredient.

### BACKGROUND ART

Polyphosphoric acid is originally contained in tissues and cells of many species and is a substance always synthesized in vivo (see H. C. Schroder et al., Inorganic polyphosphate in eukaryotes: Enzymes, metabolism and function, Progress in Molecular and Subcellular Biology, Vol. 23, 45-81, 1999). Furthermore, the safety of polyphosphoric acid for living bodies has long been known. Polyphosphoric acid is known to be a biodegradable substance that is degraded in vivo to result in atoxic phosphoric acid. The physiological functions of polyphosphoric acid remain almost unknown. However, through our series of studies on such polyphosphoric acid, it has been discovered that polyphosphoric acid has a function of stabilizing physiologically active proteins such as cell growth factors (e.g., FGF) and to control cellular physiological activity. Specifically, cultured-cell-growth-promoting action, tissue-regeneration-promoting action (see JP Patent Publication (Kokai) No. 2000-069961 A; T. Shiba et al., Modulation of Mitogenic activity of fibroblast growth factors by inorganic polyphosphate, The Journal of Biological Chemistry, Vol. 278, pp. 26788-26792, 2003), calcification-promoting action, and bone-differentiation-induction-promoting action (see JP Patent Publication (Kokai) No. 2000-79161 A) have been confirmed. As a result of further studies, for effective exertion of the tissue-regeneration-promoting action of polyphosphoric acid, it has been proposed to prepare a complex of a polyphosphoric acid and collagen (see JP Patent Publication (Kokai) No. 2004-000543 A). Furthermore, polyphosphoric acid is known to have wide-ranging effects such as an antifungal effect, discoloration prevention, vitamin C degradation prevention, anti-corrosion and anti-blackening for cans, taste improvement, and anti-turbidity. Polyphosphoric acid is also utilized as a food additive for soy sauce, juices, canned foods, and the like.

“Cytokine” is a general term for proteins that are released from leukocytes, macrophages, or the like in response to external stimulation, such as infection and tissue damage. These proteins bind to specific receptors (cytokine receptors) on the cell membrane surface. They play important roles in maintenance of in vivo homeostasis such as control of cell growth and differentiation and immune response, cell-to-cell information transmission, induction of inflammatory reaction, and anti-tumor action. There are many types of cytokine, including interferon (IFN), interleukin (IL), tumor necrosis factor (TNF), colony stimulating factor (CSF), and the like. Cytokines are largely classified in terms of function into immunoregulatory cytokines regulating immune reactions, such as IL-1, IL-2, IL-4, and IL-5, and inflammatory cytokines inducing inflammatory reactions, such as IL-6 and TNF-α. Cytokines regulate the production of each other and form a network to amplify the functions of each other. For example, IL-1, IL-6, and TNF-α mutually enhance their production of each other. In contrast, IL-4 and IL-10 suppress the secretion of the aforementioned inflammatory cytokines from mononuclear leukocytes.

Inflammation is an in vivo reaction against various forms of invasion. Moreover, inflammatory reactions are involved in the causes of various diseases or progressions of symptoms. Regarding diseases that have not been conventionally classified as inflammatory diseases, such as arteriosclerosis and Alzheimer's disease, it has been revealed by recent molecular biological studies that inflammatory reactions play an important role in the onset process of such a disease. Many mediators are involved in an inflammatory reaction. Mediators, for example, amines such as histamine and serotonin, kinins such as bradykinin, complement components, leukotriene, and prostaglandin are known. Recently, the importance of humoral factors such as cytokines and chemokines in the induction and control of inflammatory reactions has been revealed. For example, it is understood that various cytokines including inflammatory cytokines such as IL-1, IL-2, IL-6, IL-8, TNF-α, IL-α, and IL-β and leukocytotactic cytokines such as MCP-1 are involved in establishment of pathological conditions of inflammation. Furthermore, autoimmune disease is a pathological condition resulting from excessive immune response to autoantigens. Cytokines are also deeply involved in the formation of such pathological conditions.

However, suppression of cytokine production by polyphosphoric acid and the anti-inflammatory effect of polyphosphoric acid have never been reported.

An object of the present invention is to provide an anti-inflammatory agent and an anti-inflammatory medical material having a good anti-inflammatory effect and high levels of safety.

### SUMMARY OF THE INVENTION

As a result of intensive studies to achieve the above object, we have discovered that a biocompatible polyphosphoric acid with high levels of safety significantly suppresses the production of inflammatory cytokines and exerts an anti-inflammatory effect, thereby completing the present invention.

The present invention encompasses the following inventions.
(1) An anti-inflammatory agent, which contains a polyphosphoric acid as an active ingredient.
(2) The anti-inflammatory agent of (1) above, wherein a polyphosphoric acid is 1 type of linear phosphoric acid represented by the following general formula:
   Hₙ₊₂(PₙO₃ₙ₊₁)
   (wherein, n denotes an integer between 3 and 800) or a mixture of 2 or more types of such linear phosphoric acid.
(3) The anti-inflammatory agent of (2) above, wherein n in the formula is an integer between 50 and 150.
(4) The anti-inflammatory agent of any one of (1) to (3) above, wherein the polyphosphoric acid is a polyphosphate.
(5) The anti-inflammatory agent of any one of (1) to (4) above, which is used for treating and/or preventing inflammation of skin or mucous membrane.
(6) The anti-inflammatory agent of (5) above, wherein the inflammation of skin or mucous membrane is caused by a pathogenic bacterium, immune reaction, or injury.
(7) The anti-inflammatory agent of (6) above, wherein the pathogenic bacterium is an noxious intraoral bacterium and inflammation is suppressed by preventing the growth of such bacterium.
(8) The anti-inflammatory agent of (1) to (4) above, which is used for treating and/or preventing a disease caused by enhanced production of an inflammatory cytokine.
(9) The anti-inflammatory agent of (8) above, wherein the disease caused by enhanced production of an inflammatory cytokine is a disease selected from the group consisting of cancer, autoimmune disease, allergic disease, and inflammatory disease.
(10)An anti-inflammatory medical material, which contains the anti-inflammatory agent of any one of (1) to (4) above.
(11) The anti-inflammatory medical material of(10) above, wherein a polyphosphoric acid is 1 type of linear phosphoric acid represented by the following general formula:
   Hₙ₊₂(PₙO₃ₙ₊₁)
   (wherein n denotes an integer between 3 and 800) or a mixture of 2 or more types of such linear phosphoric acid.
(12) The anti-inflammatory medical material of (11) above, wherein n in the formula is an integer between 50 and 150.
(13) The anti-inflammatory medical material of any one of (10) to (12) above, wherein the polyphosphoric acid is a polyphosphate.

### BRIEF DESCRIPTION OF THE DRAWINGS

FIG. 1 shows the IL-1β production amount at each time point when human neutrophils were treated with GMDP and/or the polyphosphoric acids at different concentrations. In this figure, polyphosphoric acid concentrations are obtained by conversion using units of phosphoric acid residue, and the molecular weight thereof is approximately 102 g per mol.

FIG. 2 shows photographs of stained tissues of a treated group (treated with polyphosphoric acid) and of a comparison group (treated with phosphoric acid). “E” denotes the epithelial tissue and “Od” denotes the dentin. Portions enclosed with circles indicate the treated sites and the peripheries thereof.

FIG. 3(A) shows the effect of the polyphosphoric acid to suppress the growth of S. mutans.

FIG. 3(B) shows the effect of the polyphosphoric acid to suppress the growth of P. gingivalis.

FIG. 4 shows a wound model.

FIG. 5 shows photographs of stained tissues of (A) a treated group (treated with polyphosphoric acid) and of (B) a comparison group (treated with phosphoric acid) on day 3 after operation. In this figure, a-1-L and a-2-L are magnified photographs of “a,” a-1-L and a-2-L are images of the stained tissues from a different visual field (highly magnified images), b-1-L and b-2-L are magnified photographs of “b,” and b-1-L and b-2-L are images of the stained tissues from a different visual field (highly magnified images).

FIG. 6 shows photographs of stained tissues of (A) a treated group (treated with polyphosphoric acid) and of (B) a comparison group (treated with phosphoric acid) on day 7 after operation. In this figure, a-1-L and a-2-L are magnified photographs of “a,” a-1-L and a-2-L are images of the stained tissues from a different visual field (highly magnified images), b-1-L and b-2-L are magnified photographs of “b,” and b-1-L and b-2-L are images of the stained tissues from a different visual field (highly magnified images).

The present invention will be described more specifically below. This application claims a priority of Japanese patent application No. 2003-048460 filed on Feb. 26, 2003, and encompasses the content described in the specification and/or drawings of this patent application

The polyphosphoric acid used in the present invention is typically a linear polyphosphoric acid having a structure wherein two or more PO₄ tetrahedrons share a apex oxygen atom and are linearly linked as a result of dehydrocondensation of an orthophosphoric acid. Such a polyphosphoric acid may also be a polyphosphoric acid having side chains in which organic groups are introduced, a cyclic polyphosphoric acid, or a polyphosphoric acid (ultra polyphosphoric acid) that is a branched phosphoric acid polymer.

A polyphosphoric acid particularly preferably used in the present invention is 1 type selected from linear phosphoric acids represented by the general formula:
Hₙ₊₂(PₙO₃ₙ₊₁)
(wherein n denotes an integer between 3 and 800) or is a mixture of 2 or more types selected from such linear phosphoric acids.

“n” in the general formula is an integer between 3 and 800, preferably between 30 and 500, and more preferably between 50 and 150.

In addition, a polyphosphoric acid having a chain length of 1000 or more is not preferable because it has not been confirmed whether such a polyphosphoric acid is present in the form of an aqueous solution and such a polyphosphoric acid is thought to be slightly soluble to water. Furthermore, the chain length of polyphosphoric acid in vivo is approximately 800. Thus, a polyphosphoric acid having a chain length of 800 or less is thought to have high efficiency relating to various physiological functions in vivo (K. D. Kumble and A. Kornberg, Inorganic polyphosphate in mammalian cells and tissues, The Journal of Biological Chemistry, Vol. 270, pp. 5818-5822, 1995).

Furthermore, in the present invention, a polyphosphate having a molecular structure wherein hydrogen of a hydroxyl group of the above polyphosphoric acid is substituted with a metal may be used. Examples of such metal include sodium, potassium, calcium, and magnesium.

A polyphosphoric acid or a salt thereof to be used in the present invention may be of 1 type or a mixture of a plurality of types of the same. Examples of a plurality of types of polyphosphoric acid or salts thereof include polyphosphoric acids having different polymerization degrees or salts thereof, polyphosphoric acids having different molecular structures or salts thereof, and polyphosphates having different metal ions. Furthermore, both a polyphosphoric acid and a salt thereof may be included.

The above polyphosphoric acid can be produced by a generally employed production method, such as a method that involves heating a phosphoric acid and a method that involves adding and dissolving phosphorus pentoxide to a phosphoric acid.

Moreover, particularly a medium- or long-chain polyphosphoric acid with a chain length of 20 or more can be produced by the following method that we have developed. First, hexametaphosphate is dissolved in water to a level of 0.1% to 10% by weight, and preferably 10% by weight. To the hexametaphosphate aqueous solution, 87% to 100% ethanol, and preferably 96% ethanol, is added in a volume that is one tenth to one third of the entire mixed solution comprised of the hexametaphosphate aqueous solution and ethanol; that is, to achieve a volume ratio of the hexametaphosphate aqueous solution to ethanol that ranges from 2:1 to 9:1. The mixed solution is sufficiently agitated. The resulting precipitate is separated from aqueous solution components by a separation method such as centrifugation or filter filtration. But the method is not limited thereto. The thus separated precipitate is a medium- or long-chain polyphosphoric acid. The polyphosphoric acid is subsequently washed with 70% ethanol and then dried. The average chain length of polyphosphoric acids obtained by such a separation procedure ranges from 60 to 70 and almost no short-chain polyphosphoric acids having chains of 10 or less are included. Hence, the molecular weight distribution ranges from approximately 10 to 150 (based on the number of phosphoric acid residues).

The polyphosphoric acid content of the anti-inflammatory agent of the present invention is not specifically limited and may be, for example, 0.001% to 20% by weight, preferably 0.01% to 10% by weight, more preferably 0.1% to 5% by weight, and most preferably 0.2% to 2% by weight.

A polyphosphoric acid or a salt thereof may be formulated alone into, or mixed with pharmacologically and pharmaceutically acceptable additives and then formulated into an oral and/or parenteral pharmaceutical preparation. Such pharmaceutical preparations may exist in various dosage forms including tablets, powder, granules, fine grains, capsules, solutions for internal use (e.g., suspensions, syrups, and emulsions), solutions for external use (e.g., agents for infusion, collutorium/mouth washes, air spray/aerosol, inhalants, and liniments), ointments, injections, drops, and suppositories.

As pharmacologically and pharmaceutically acceptable additives, for example, excipients, disintegrating agents or disintegration aids, binders, lubricants, coating agents, dye, diluents, bases, solvents or solubilizing agents, isotonizing agents, pH adjusters, stabilizers, propellants, and adhesives can be used.

For pharmaceutical preparations for oral administration, as additives, for example, excipients such as glucose, lactose, D-mannitol, starch, or crystalline cellulose; disintegrating agents or disintegration aids such as carboxymethylcellulose, starch, or carboxymethylcellulose calcium; binders such as hydroxypropylcellulose, hydroxypropylmethylcellulose, polyvinylpyrrolidone, or gelatine; lubricants such as magnesium stearate or talc; coating agents such as hydroxypropylmethylcellulose, saccharose, polyethylene glycol or titanium oxide; and bases such as vaseline, liquid paraffin, polyethylene glycol, gelatine, Kaolin, glycerine, purified water, or hard fat can be used.

For pharmaceutical preparations for parenteral administration, solvents such as distilled water, physiological saline, ethanol, glycerine, propylene glycol, macrogol, alum solution, or plant oil; isotonizing agents such as glucose, sodium chloride, or D-mannitol; and pH adjusters such as inorganic acid, organic acid, inorganic nucleotide or organic nucleotide can be used.

Furthermore, the anti-inflammatory agent of the present invention can also be used by mixing a polyphosphoric acid with existing antibiotics or an anti-inflammatory agent to obtain stronger anti-inflammatory action. In this case, examples of a drug to be mixed therewith include tetracycline, quinolone antiinflammatory agent, chloramphenicol, and penicillin antibiotic.

The active ingredient of the anti-inflammatory agent of the present invention, that is, polyphosphoric acid, has action to suppress the production of inflammatory cytokines and action to suppress the growth of noxious intraoral bacteria. Thus, the anti-inflammatory agent of the present invention is effective as a remedy for treating and/or preventing inflammation of skin or mucous membrane or treating and/or preventing diseases caused by enhanced production of inflammatory cytokines.

In the present invention, “inflammation of skin or mucous membrane” means inflammation of skin or mucous membrane, and particularly oral mucous membrane, caused by pathogenic bacteria, immune reactions, injuries, or the like. Specifically, examples of such inflammation include, but are not limited to: inflammation due to pathogenic bacteria such as noxious intraoral bacteria (e.g., bacteria associated with dental caries (e.g., Streptococcus mutans) and bacteria associated with periodontal disease (e.g., Porphyromonas gingivalis)), acne bacilli, and staphylococci; inflammation due to wounds, burns, or the like; inflammation within narrow areas due to atopic dermatitis or pollinosis; and inflammation within wide areas due to rejection at the time of organ transplantation.

Furthermore, examples of “diseases caused by enhanced production of inflammatory cytokines” include, but are not limited to: cancer such as gastric cancer, large bowel cancer, breast cancer, lung cancer, esophageal cancer, prostate cancer, liver cancer, kidney cancer, bladder cancer, skin cancer, uterine cancer, brain tumor, osteosarcoma, and myeloma; autoimmune diseases such as chronic rheumatism, multiple sclerosis, myasthenia gravis, thyroiditis, polymyositis, pachyderma, dermatomyositis, polyarteritis nodosa, systemic erythematodes, Behcet's disease, and Basedow's disease; allergosis such as bronchial asthmatic attack, atopic dermatitis, allergic rhinitis, pollinosis, and urticaria; inflammatory diseases such as inflammatory bowel disease (IBD), colitis ulcerosa, Crohn's disease, ichorrhemia, arthritis, uveitis, keratitis, and SIRS (systemic inflammatory response syndrome).

Furthermore, the inflammatory cytokine means at least one type or more of cytokine selected from IL-1, IL-2, IL-4, IL-5, IL-6, IL-8, IL-13, IL-16, IL-17, IL-18/IGIF, IL-12p35, IL-12p40, MIF, IL-1α, IL-1β, GM-CSF, TNF-α, TGF-β, EGF, FGF, PDGF, IFN-α, IFN-β, IFN-γ, MCP-1, and PANTES.

A method for administering the anti-inflammatory agent of the present invention may be either an oral or parenteral method. For example, in the case of a liniment and/or ointment, the agent may be directly applied to the portion of the periodontal tissue, stomatitis, dermatitis, or hemorrhoid. In the case of air spray or the like, the agent may be administered by intranasal, intraoral, or intratracheal spraying. Alternatively, the agent may also be applied as an eye drop to eyes.

The dose of the anti-inflammatory agent of the present invention is not specifically limited and is appropriately adjusted depending on age, sex, symptoms, weight, or the like of a patient. For example, when the agent is administered orally to an adult, the agent is administered once or several times a day. The dose of the agent is 10 to 1000 mg/kg body weight/day, and preferably 50 to 500 mg/kg body weight/day.

Furthermore, the anti-inflammatory agent of the present invention may be compounded not only in a remedy, but also in compositions such as quasi-drugs or cosmetics used for the purpose of imparting an anti-inflammatory effect. Examples of quasi-drugs, cosmetics, or the like include lotions, emulsions, creams, facial washes, toothpastes, mouth washes, and gargles. To these compositions, drugs that are generally used in the art, such as surfactants, moisturizing agents, ultraviolet absorbers, aromatic chemicals, or antiseptic agents may be appropriately compounded therein.

Moreover, as described above, polyphosphoric acid itself has tissue regeneration-promoting action (JP Patent Publication (Kokai) No. 2000-69961 A) and bone-regeneration-promoting action (JP Patent Publication (Kokai) No. 2000-79161 A). Polyphosphoric acid can be used to impart anti-inflammatory property to medical materials other than polyphosphoric acid, such as biocompatible materials or scaffolds for regenerative medicine by mixing it into such medical materials or coating such medical materials therewith. Examples of such materials include bioceramics (e.g., alumina, titanium oxide, zirconia, carbon, apatite, A-W glass ceramics, calcium phosphate glass ceramics, and tribasic calcium phosphate (TCP)), natural polymer materials (e.g., collagen, gelatine, chitin, chitosan, cellulose, and hyaluronic acid), medical metal materials (e.g., titanium and titanium alloy), and synthetic polymer materials (e.g., glycol/dicarboxylic acid materials, polyester carbonate, polycaprolactone, polylactic acid, and polyglycolic acid). The thus obtained anti-inflammatory medical materials can be safely used in vivo as medical appliances such as artificial internal organs, artificial skin, artificial joints, artificial dentures, artificial dental roots, artificial vessels, artificial bone, and surgical sutures without inducing any inflammation in vivo.

### BEST MODE OF CARRYING OUT THE INVENTION

The present invention will be hereafter described in detail by referring to examples, but the invention is not limited by these examples.

### PRODUCTION EXAMPLE

### Production of Polyphosphoric Acid (Medium- or Long-Chain Polyphosphoric Acid

20 g of sodium hexametaphosphate (based on the food additive standards) was dissolved in 200 ml of purified water, to which 32 ml of 96% ethanol was then gradually added. The solution was agitated well and left to stand at room temperature for approximately 30 minutes, and then subjected to centrifugation (10,000×g, 20 minutes, and 25° C.), thereby separating aqueous solution components from the precipitate. The aqueous solution components were discarded. 70% ethanol was added to the collected precipitate, followed by washing and vacuum drying. In this manner, 9.2 g of medium- and long-chain (average chain length of 60 or more) polyphosphates was obtained as a precipitate (a yield of 46.0%).

### EXAMPLE 1

### Suppression of Inflammatory Cytokine Production with Polyphosphoric Acid

To confirm the effect of suppressing inflammatory cytokine production with the polyphosphoric acid obtained according to the above production example, an experiment was conducted to observe IL-1β production using human neutrophils. Neutrophils were separated from 25 ml of human blood and then suspended in a Dulbecco's Modified Eagle's Medium. To the separated neutrophils, 30 μg of GMDP, which is a glycopeptide adjuvant [N-Acetyl-D-Glucosaminyl-β(1-4)-N-acetylmuramyl-L-alanyl-D-isoglutamine], or the polyphosphoric acids at different concentrations (0.2 mM, 1 mM, and 5 mM), and both GMDP and the polyphosphoric acids at different concentrations were added. Treatment was carried out for maximum of 5 hours at 37° C. Treatment was similarly carried out for a comparison group to which none had been added and for comparison groups to which only the polyphosphoric acids at different concentrations were added. The IL-1β production amount at each time point was measured using an ELISA Kit.

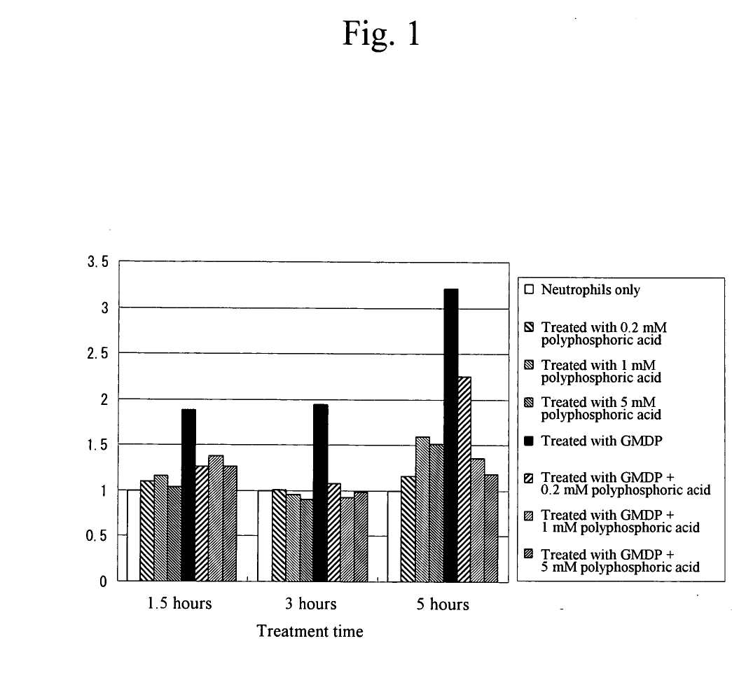

FIG. 1 shows the IL-1β production amount at each time point when human neutrophils were treated with GMDP and the polyphosphoric acids at different concentrations. In the case of treatment with GMDP alone, the IL-1β production amount increased with time. However, in the case of treatment with both GMDP and the polyphosphoric acids, compared with treatment with GMDP alone, the IL-1β production amount was suppressed to a level similar to that of the comparison group (untreated neutrophils) until 3 hours after treatment. Even 5 hours after treatment, the IL-1β production amount decreased depending on the concentrations of the polyphosphoric acids. Moreover, in the case of treatment with the polyphosphoric acids alone, the IL-1β production amount was almost the same at any polyphosphoric acid concentration as that in the case of the comparison group (untreated neutrophils). This indicates that enhanced IL-1β production by neutrophils, which is induced by GMDP, is significantly suppressed by the polyphosphoric acid.

### EXAMPLE 2

### Suppression of Periodontal Tissue Inflammation with Polyphosphoric Acid

To confirm the anti-inflammatory effect of the polyphosphoric acid obtained according to the above production example, an experiment using rats was conducted wherein the inflammatory conditions of tissues were observed. Male Wister rats (8-week-old; 10 rats in total) were anesthetized, and then the periosteum of the alveolar part of maxillary first molar tooth was stripped. Portions of approximately 2 mm were eliminated from the buccal alveolar apexes of the maxillary first molar tooth and of the maxillary second molar tooth, using a ½ round bar, thereby forming artificial periodontal pockets (gingival crevices). In the case of the treated group (5 rats), approximately 0.1 ml of 1% polyphosphoric acid solution was injected into each gingival crevice using a syringe. Furthermore, in the case of the comparison group (5 rats), only a 1% phosphate buffer was injected. This injection procedure was carried out every day for 10 days from the day following the operation for the preparation of gingival crevices.

Rats that had been treated for a certain time period were euthanized under inhalation anesthesia (ether). The maxilla bones were amputated, and then the tissues were fixed for 1 day by immersion using 10% neutral buffered formalin (pH 7.4). Subsequently, the resultants were subjected to acid demineralization at room temperature for approximately 2 days. After completion of demineralization, the samples were trimmed by excision at the second molar tooth, and then paraffin-embedded with their cut surfaces positioned downward. Thus, tissue sections were prepared, subjected to HE staining, and then observed.

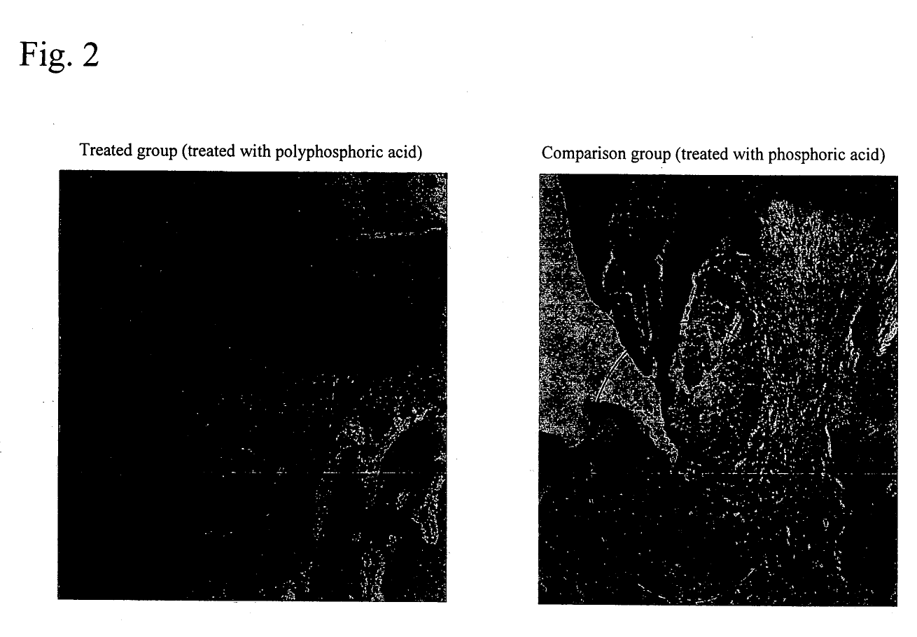

FIG. 2 shows the images of stained tissue samples selected from the treated group (treated with polyphosphoric acid) and the comparison group (treated with a phosphate buffer). In this figure, “E” denotes the epithelial tissue and “Od” denotes the dentin. Portions enclosed with circles indicate the treated sites and the peripheries thereof Almost no inflammatory cells were observed in the peripheries of the treated sites of the group treated with the polyphosphoric acid. However, many inflammatory cells were observed in the peripheries of the treated sites of the comparison group (the group treated with phosphoric acid) and bacterial reproduction was also confirmed. This indicates that polyphosphoric acid suppresses the growth of bacteria associated with periodontal diseases and significantly suppresses inflammation.

### EXAMPLE 3

### Test of Suppression of Intraoral Bacteria Growth with Polyphosphoric Acid

A test of suppression of bacterial growth with the polyphosphoric acid obtained according to the above production example was conducted using intraoral bacteria, Streptococcus mutans (S. mutans) and Porphylomonas gingivalis (P gingivalis). The S. mutans JC2 strain was anaerobically cultured at 37° C. using Heart infusion media. For the treated groups, polyphosphoric acid was added to the culture solutions at different concentrations (0%, 0.06%, and 0.5%). For comparison groups, phosphate buffers were added at different concentrations (0%, 0.06%, and 0.5%) to culture solutions. The bacteria were cultured for a maximum of 2 days. The bacterial growth was observed with time by measuring absorbance at 595 nm. In the meantime, the P. gingivalis ATCC33277 strain was anaerobically cultured using brain-heart-infusion media at 37° C. for the same time period as employed above by adding polyphosphoric acid at different concentrations (0%, 0.015%, 0.03%, 0.06%, 0.12%, 0.25%, and 0.5%) to culture solutions. Bacterial growth was observed over time by measuring absorbance at 595 nm.

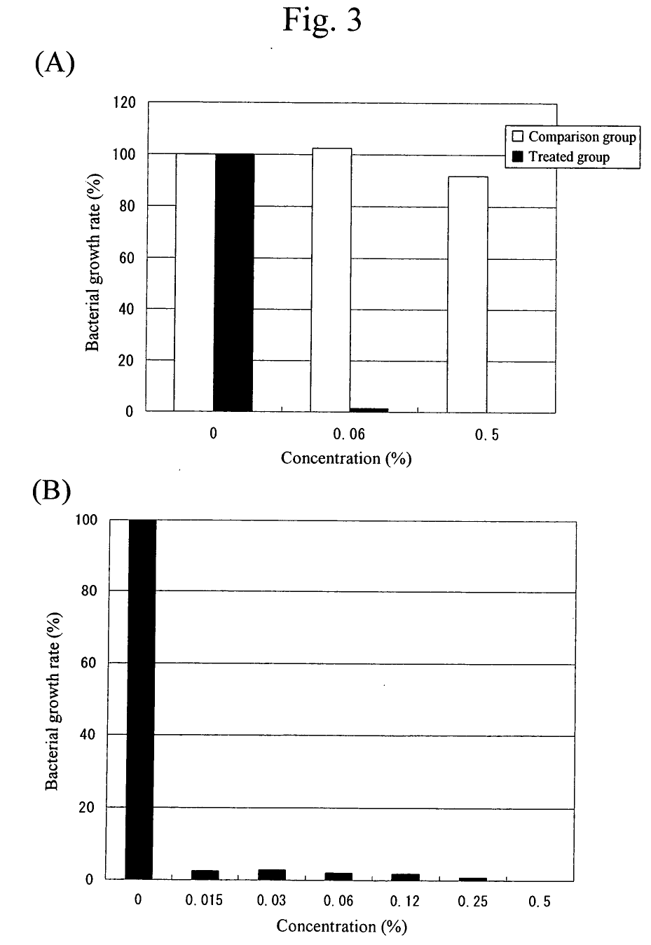

FIG. 3(A) shows the results of the growth of S. smutans in the treated groups and the comparison groups 24 hours after treatment and FIG. 3(B) shows the results of the bacterial growth of P. gingivalis in the treated groups and the comparison groups 48 hours after treatment. In the case of S. mutans, no large changes were observed in bacterial growth even at a phosphoric acid concentration of 0.5%, but significantly suppressed bacterial growth was observed at a polyphosphoric acid concentration of 0.06%. Also in the case of P. gingivalis, significantly suppressed bacterial growth was observed at a polyphosphoric acid concentration of 0.01%. This indicates that polyphosphoric acid suppressed the growth of the intraoral bacteria.

### EXAMPLE 4

### Test of Suppression of Inflammation at Wound Site with Polyphosphoric Acid

A test of anti-inflammatory action at rat wound sites using the polyphosphoric acid obtained according to the above production example was conducted.

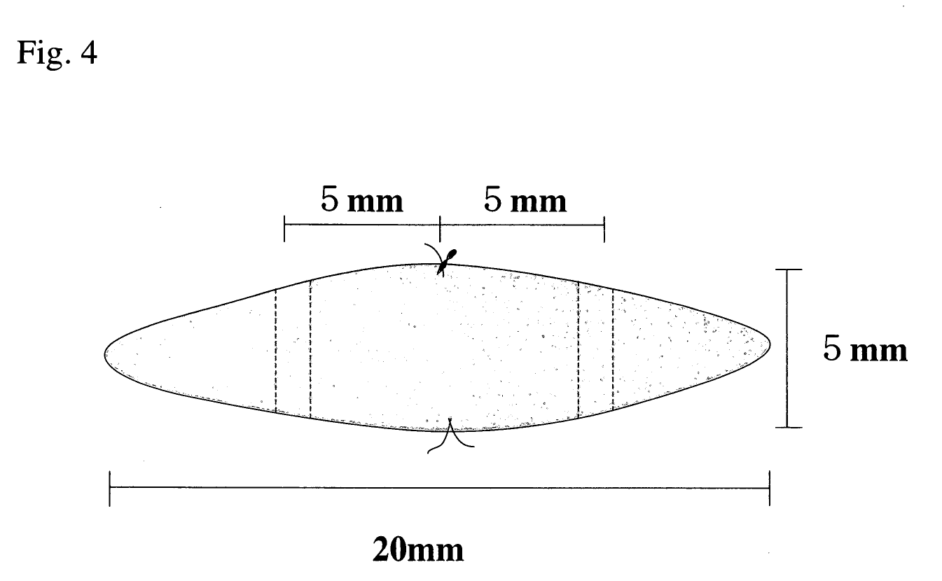

The dorsum of each 6-week-old male Wistar rat was shaved under ether anesthesia, and then a 20-mm incision was made along the long axis of the body with a depth reaching to the fascia. Both ends of the center of the wound were stitched to the fascia using a piece of string so as to produce a 5-mm-wide spindle-shaped wound model (FIG. 4). A 1% polyphosphoric acid solution was locally injected 5 days a week into each wound of the rats in the treated group. A 1% phosphate buffer was locally injected 5 days a week into each wound of the rats in the comparison group. The rats were euthanized on days 3 and 7. Tissue located in the area extending from 5 mm to the left of and 5 mm to the right of the wound center was obtained by excising skin on the fascia as shown in FIG. 4. The tissues were subjected to HE staining and then histopathological observance was carried out.

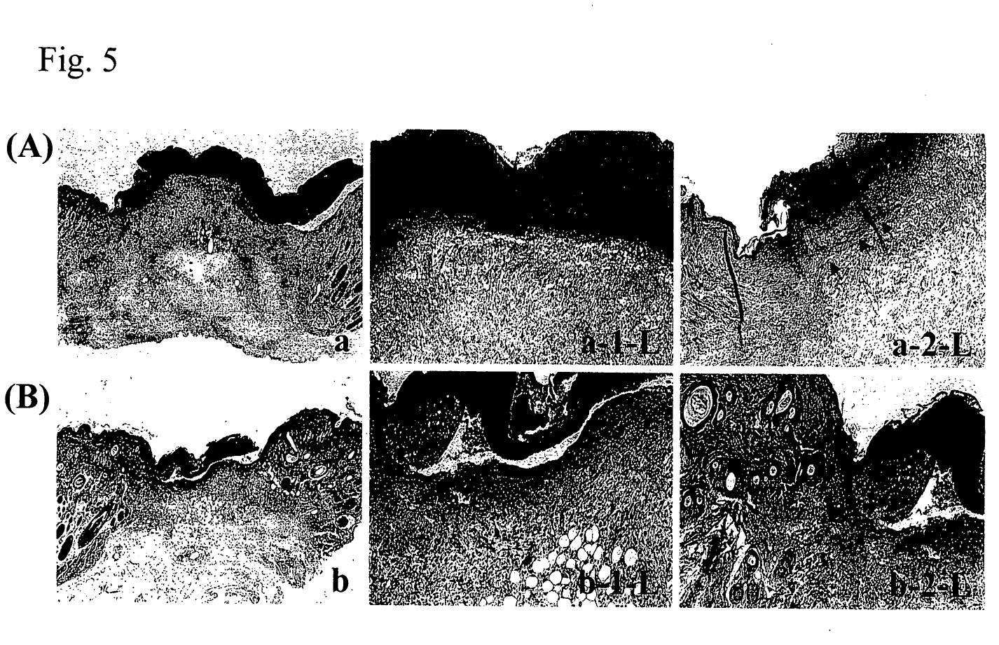

FIG. 5 shows the images of stained tissue samples selected from (A) the group treated with the polyphosphoric acid and (B) the comparison group treated with a phosphate buffer on day 3 after operation. In the case of the wound surfaces of the comparison group, infiltration caused by inflammatory cells including mainly neutrophils, was still observed (see “a” and a-1-L, which is a magnified photograph of “a”). However, in the case of the group treated with polyphosphoric acid, the proportion of neutrophils decreased and lymphocytes and macrophages emerged. Overall, infiltration by inflammatory cells decreased and many spindle-shaped fibroblasts that were relatively rich in cytoplasm were observed (see “b” and b-1-L, which is a magnified photograph of “b”). Furthermore, in the case of the comparison group, only slight extension (indicated with arrows) of the epithelia was observed (a-2-L). However, in the case of the group treated with the polyphosphoric acid, significant extension (indicated with arrows) of the epithelia toward the centers of the wounds was observed (b-2-L).

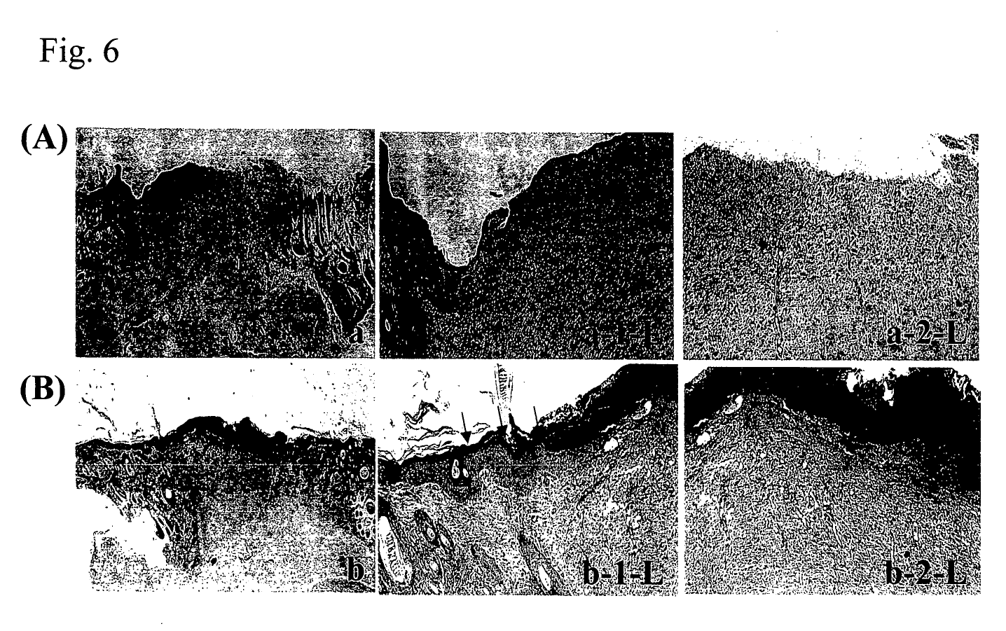

FIG. 6 shows the images of stained tissue samples selected from (A) the group treated with the polyphosphoric acid and (B) the comparison group treated with a phosphate buffer on day 7 after operation. Both (A) and (B) groups lacked the epithelia of the centers of the wounds. At the cut ends of the wounds, compared with the comparison group (see “a” and a-1-L, which is a magnified photograph of “a”), the extension (indicated with arrows) of the epithelia was promoted in the group treated with the polyphosphoric acid (see “b” and b-1-L, which is a magnified photograph of “b”). At the centers of the wounds in the case of the comparison group, the deep part had still been infiltrated by inflammatory cells (a-2-L). However, in the case of the group treated with the polyphosphoric acid, inflammation remained slight and partial on the upper parts, repair took place in the deep parts via fibrous connective tissues, and organification proceeded (b-2-L).

Based on the above results, in the case of the group treated with the polyphosphoric acid, compared with the comparison group, infiltration by inflammatory cells started to decrease on day 3 after operation, organification of subcutaneous tissues was promoted, the epithelia extended early, and the wound surfaces were almost completely epithelialized on day 7 after operation. As described above, it was revealed that the polyphosphoric acid also significantly suppresses inflammation in skin tissue and promotes the repair and/or regeneration thereof.

All publications, patents and patent applications cited herein are incorporated herein by reference in their entirety.

### INDUSTRIAL APPLICABILITY

According to the present invention, an anti-inflammatory agent having action to suppress the production of inflammatory cytokines is provided. The anti-inflammatory agent of the present invention can be applied by directly coating inflammatory sites of skin, mucous membrane, and the like therewith. In particular, the anti-inflammatory agent exerts an anti-inflammatory effect by suppressing the growth of noxious intraoral bacteria, such as bacteria associated with dental caries and bacteria associated with periodontal diseases. Furthermore, the anti-inflammatory agent is effective for treating and/or preventing diseases caused by enhanced production of inflammatory cytokines in cases such as cancer and autoimmune diseases.

1. An anti-inflammatory agent, which contains a polyphosphoric acid as an active ingredient.

2. The anti-inflammatory agent of claim 1, wherein a polyphosphoric acid is 1 type of linear phosphoric acid represented by the following general formula:
Hₙ₊₂(PₙO₃ₙ₊₁)
(wherein, n denotes an integer between 3 and 800) or a mixture of 2 or more types of such linear phosphoric acid.

3. The anti-inflammatory agent of claim 2, wherein n in the formula is an integer between 50 and 150.

4. The anti-inflammatory agent of any one of claims 1 to 3, wherein the polyphosphoric acid is a polyphosphate.

5. The anti-inflammatory agent of any one of claims 1 to 3, which is used for treating and/or preventing inflammation of skin or mucous membrane.

6. The anti-inflammatory agent of claim 5, wherein the inflammation of skin or mucous membrane is caused by a pathogenic bacterium, immune reaction, or injury.

7. The anti-inflammatory agent of claim 6, wherein the pathogenic bacterium is an noxious intraoral bacterium and inflammation is suppressed by preventing the growth of such bacterium.

8. The anti-inflammatory agent of any one of claims 1 to 3, which is used for treating and/or preventing a disease caused by enhanced production of an inflammatory cytokine.

9. The anti-inflammatory agent of claim 8, wherein the disease caused by enhanced production of an inflammatory cytokine is a disease selected from the group consisting of cancer, autoimmune disease, allergic disease, and inflammatory disease.

10. An anti-inflammatory medical material, which contains a polyphosphoric acid.

11. The anti-inflammatory medical material of claim 10, wherein the polyphosphoric acid is 1 type of linear phosphoric acid represented by the following general formula:
Hₙ₊₂(PₙO₃ₙ₊₁)
(wherein n denotes an integer between 3 and 800) or a mixture of 2 or more types of such linear phosphoric acid.

12. The anti-inflammatory medical material of claim 11, wherein n in the formula is an integer between 50 and 150.

13. The anti-inflammatory medical material of any one of claims 10 to 12, wherein the polyphosphoric acid is a polyphosphate.

14. The anti-inflammatory agent of claim 4, which is used for treating and/or preventing inflammation of skin or mucous membrane.

15. The anti-inflammatory agent of claim 14, wherein the inflammation of skin or mucous membrane is caused by a pathogenic bacterium, immune reaction, or injury.

16. The anti-inflammatory agent of claim 14, wherein the pathogenic bacterium is an noxious intraoral bacterium and inflammation is suppressed by preventing the growth of such bacterium.

17. The anti-inflammatory agent of claim 4, which is used for treating and/or preventing a disease caused by enhanced production of an inflammatory cytokine.
